(19) United States
(12) Patent Application Publication
(10) Pub. No.: US 2021/0330644 A1
(43) Pub. Date: Oct. 28, 2021

(54) COMBINATION THERAPY FOR TREATING METASTATIC PROSTATE CANCER

(71) Applicant: Progenics Pharmaceuticals, Inc, New York, NY (US)

(72) Inventor: Vivien Wong, Scarsdale, NY (US)

(73) Assignee: Progenics Pharmaceuticals, Inc, New York, NY (US)

(21) Appl. No.: 17/227,024

(22) Filed: Apr. 9, 2021

Related U.S. Application Data
(63) Continuation of application No. PCT/US2019/055931, filed on Oct. 11, 2019.
(60) Provisional application No. 62/842,136, filed on May 2, 2019.
(60) Provisional application No. 62/744,400, filed on Oct. 11, 2018.

(51) Int. Cl. A61K 31/4166; A61K 51/04; A61P 35/04

(52) U.S. Cl. CPC A61K 31/4166; A61P 35/04; A61K 51/0402

(57) ABSTRACT

Provided herein are compositions and related methods for treating and/or identifying patients likely to respond to treatments for prostate cancer.

### RELATED APPLICATIONS

This application claims the benefit of priority under 35 U.S.C. § 119 to U.S. Provisional Application No. 62/744,400 filed Oct. 11, 2018 and U.S. Provisional Application No. 62/842,136 filed May 2, 2019, the entire contents of each of which are incorporated herein by reference.

### BACKGROUND OF THE INVENTION

Prostate cancer is the second most common form of cancer affecting men in the United States: an estimated one in seven men will be diagnosed with prostate cancer in his lifetime. The American Cancer Society estimates that each year approximately 164,609 new cases of prostate cancer will be diagnosed, and about 26,730 men will die of the disease. Approximately 2.9 million men in the U.S. currently count themselves among prostate cancer survivors.

Therapies have been shown to prolong life in subjects; however, the survival benefits are at times only modest, and generally, prostate cancers eventually become hormone refractory and progress. Additionally, hormone-refractory prostate cancer or androgen-independent prostate cancer has proven to be largely resistant to conventional chemotherapy. New therapies are needed to expand therapeutic options and/or improve survival for subjects with prostate cancer including those with metastatic prostate cancer; metastatic, castration-resistant prostate cancer (mCRPC) as well as prostate cancer that has progressed despite prior treatment.

### SUMMARY OF THE INVENTION

In retrospective analyses, patients treated with enzalutamide following progression under abiraterone therapy of any duration, reported variable proportions of patients with >50% decline in PSA from baseline, ranging from 30-51%. A study on mCRPC patients who received all available therapies at that time reported a ≥50% PSA response of 70.6% after 1^{st} therapy.

The majority of data demonstrate limited benefit from enzalutamide in abiraterone-resistant patients. Resistance to abiraterone and subsequent response to enzalutamide may, for example, be caused by androgen receptor (AR) gain of function mutants enabling the AR to be activated by nonandrogenic steroids that do not require CYP17A1 for synthesis. However, response and resistance mechanisms are considered to be heterogenous and evolve with selective pressure of prescribed treatments. Cross-resistance might also involve tumor steroidogenesis as preclinical data support the role of tumor steroidogenesis as a mechanism of evolution to CRPC and resistance to enzalutamide.

Cross-resistance between docetaxel and abiraterone with lower than expected PSA response rate may exist in patients treated with docetaxel following abiraterone, further narrowing treatment options for patients with mCRPC. Furthermore, recent preclinical data suggest that the pro-apoptotic effects of enzalutamide could sensitize cells to radiotherapy-induced cell death. Thus, the combination of radiotherapy and enzalutamide can be a more effective treatment paradigm for patients with mCRPC in some instances.

The population failing 1^{st} line mCRPC therapy with novel anti-androgens such as abiraterone are in need of effective alternative options and mechanisms of action. Moreover, methods for identifying patients likely to respond to these alternative option treatments are needed.

Any one of the methods and compositions provided herein can be used for treating any one the subjects described herein.

In one aspect, a method of treating a subject with prostate specific membrane antigen (PSMA)-avid prostate cancer, the method comprising administering to a subject one or more doses of I-131 1095 and administering one or more doses of enzalutamide is provided. In one embodiment, the subject's tumor PSMA avidity, prior to the administering of the one or more doses of I-131 1095 and the administering of the one or more doses of enzalutamide, is/was assessed.

In another aspect, a method of treating a chemotherapeutic-naïve subject whose castration-resistant prostate cancer has progressed despite abiraterone treatment by administering one or more doses of I-131 1.095 and administering one or more doses of enzalutamide is provided. In one embodiment, the method further comprises determining PSMA-avidity of the cancer using a radiolabeled PSMA-binding agent. In one embodiment of any one of the methods provided herein, the PSMA-binding agent is any one of the binding agents provided herein or otherwise known in the art.

In another aspect, a method of treatment management for a subject with PSMA-avid metastatic, castration resistant prostate cancer and cancer progression on prior abiraterone therapy comprising a) demonstrating the subject's tumor PSMA avidity in bone lesions and/or visceral or lymph node lesions, and b) providing a treatment of one or more doses of I-131 1095 and one or more doses of enzalutamide is provided. In one embodiment, the demonstrating the subject's tumor PSMA avidity is determined by at least one bone lesion (SUV>1.5 SUV of liver) and/or at least one visceral or lymph node lesion (that has a long diameter of >2 cm).

In any one of the methods provided herein, the method further comprises assessing tumor avidity in the subject prior to the administering of the one or inure doses of I-131 1095 and the administering of the one or more doses of enzalutamide. In any one of the methods provided herein, the assessment of tumor avidity comprises any one of the methods of such assessment provided herein.

In any one of the methods provided herein, the tumor avidity was/is assessed with [18F]DCFPyL PET/CT. In any one of the methods provided herein, the [18F]DCFPyL PET/CT indicates significant uptake (SUVmax>1×SUVmean of liver) in at least one lesion (e.g., all lesions observed in one embodiment) except where: (a) PSMA negative soft tissue lesions <1.0 cm in short axis, (b) PSMA negative lymph node lesions <1.5 cm in short axis; and/or (c) PSMA negative bone lesions with a soft tissue component <1.0 cm in short axis or without a soft tissue component of any size.

In any one of the methods provided herein, the [18F]DCFPyL PET/CT indicates significant uptake (SUV>1.5×SUV of liver) in at least one bone lesion and/or indicates significant uptake in at least one visceral or lymph node lesion that has a long diameter of >2 cm.

In any one of the methods provided herein, the subject is any one of the subjects as described herein.

In any one of the methods provided herein, the subject is any one of the subjects as defined in the Examples, such as defined with the inclusion criteria and/or the exclusion criteria in Example 1.

In any one of the methods provided herein, the subject (e.g., meets one or more or all of the following): (a) has castration-resistant prostate cancer with serum testosterone ≤50 ng/dL (1.73 nM), (b) has metastatic disease documented by bone lesions on whole body bone scan or soft tissue lesions measurable per RECIST 1.1 on CT/MRI, (c) had disease progression on prior abiraterone therapy, (d) did not receive prior taxane-based chemotherapy, (e) had prior treatment with bisphosphonates and on stable doses for ≥4 weeks prior, and (f) Eastern Cooperative Oncology Group (ECOG) performance status 0-2.

In any one of the methods provided herein, disease progression on prior abiraterone therapy is defined by meeting at least one of the following (e.g., one or more or all): (i) PSA progression defined by a minimum of two rising PSA levels at least 1 week apart, (ii) soft tissue disease progression defined by RECIST 1.1, and (iii) bone disease progression defined by two or more new lesions on bone scan.

In any one of the methods provided herein, the subject has not/does not (e.g., one or more or all of the following): (a) received any anti-tumor therapy within 4 weeks prior (not including abiraterone, gonadotropic-releasing hormone (GnRH) therapy and/or non-radioactive bone-targeted agents in some embodiments), (b) received prior chemotherapy for prostate cancer, (c) received treatment with Strontium-89, Samarium-153, Rhenium-186, Rhenium-188, Radium-223 within 6 months prior, (d) had a prior hemi-body irradiation or prior external beam radiotherapy to >25% of bone marrow, (e) had a prior PSMA-targeted radioligand therapy, (f) have impaired organ function, and (g) have hypothyroidism.

In any one of the methods provided herein, the impaired organ function is: (i) absolute neutrophil count <1500 μL, (ii) platelet count <100,000/μL, (iii) hemoglobin <9.5 g/dL, (iv) albumin <3.0 g/dL (30 g/L), (v) total bilirubin >2×ULN (not including in instances of known or suspected Gilbert's disease in some embodiments), (vi) AST and ALT>2.5×ULN, and/or (vii) serum creatinine >1.5×ULN or calculated creatinine clearance (CrCL)<30 mL/min (Cockroft-Gault equation) or on renal dialysis.

In any one of the methods provided herein, hypothyroidism is TSH>3 or 4.0 milt/I, with low Free T3 (<230 ng/dL) or Free T4 (<0.7 ng/dL).

In any one of the methods provided herein, the subject has PSMA-avid metastatic castration resistant prostate cancer (mCRPC).

In any one of the methods provided herein, the subject has PSMA-avid mCRPC and disease progression on prior antiandrogen therapy.

In any one of the methods provided herein, the prior antiandrogen therapy is prior abiraterone therapy.

In any one of the methods provided herein, a whole body bone scan of the subject is assessed at intervals to determine changes over time and/or a Bone Scan Index (BSI) is determined.

In any one of the methods provided herein, the method further comprises assessing the subject prior to the administering of the one or more doses of I-131 1095 and the administering of the one or more doses of enzalutamide, the assessing comprising (e.g., one or more or all of the following): (a) determining the subject has castration-resistant prostate cancer with serum testosterone ≤50 ng/dL (1.73 nM), (b) determining the subject has metastatic disease documented by bone lesions on whole body bone scan or soft tissue lesions measurable per RECIST 1.1 on CT/MRI, (c) determining the subject has disease progression on prior abiraterone therapy, (d) determining the subject has not had prior taxane-based chemotherapy, (e) determining the subject has had prior treatment with bisphosphonates and on stable doses for ≥4 weeks prior, and (f) determining the subject has Eastern Cooperative Oncology Group (ECOG) performance status 0-2.

In any one of the methods provided herein, the method further comprises assessing the subject prior to the administering of the one or more doses of I-131 1095 and the administering of the one or more doses of enzalutamide, the assessing comprising (e.g., one or more or all of the following): (a) determining the subject has not received any anti-tumor therapy within 4 weeks prior (not including abiraterone, gonadotropic-releasing hormone (GnRH) therapy and/or non-radioactive bone-targeted agents in some embodiments), (b) determining the subject has not received prior chemotherapy for prostate cancer, (c) determining the subject has not received treatment with Strontium-89, Samarium-153, Rhenium-186, Rhenium-188, Radium-223 within 6 months prior, (d) determining the subject has not had a prior hemi-body irradiation or prior external beam radiotherapy to >25% of bone marrow, (e) determining the subject has not had a prior PSMA-targeted radioligand therapy, (f) determining the subject does not have impaired organ function, and (g) determining the subject has not have hypothyroidism.

In any one of the methods provided herein, each dose of enzalutamide and/or each dose of I-131 1095 is/are any one of such doses provided herein. In any one of the methods provided herein, each dose of I-131 1095 is between 75-100 mCi. In any one of the methods provided herein, the I-131 1.095 dose is administered or provided every 12 weeks. In any one of the methods provided herein, the I-131 1095 dose is administered or provided every 8 weeks. In any one of the methods provided herein, each dose of enzalutamide is administered or provided orally once a day. In any one of the methods provided herein, each dose of enzalutamide is four 40 mg capsules administered or provided orally once a day. In any one of the methods provided herein, each dose of I-131 1095 is 75-1.00 mCi is administered or provided every 8 to 14 weeks and each dose of enzalutamide is four 40 mg capsules administered or provided orally once a day.

In any one of the methods provided herein, each dose of I-131 1095 is administered intravenously.

In any one of the methods provided herein, the method provides a PSA response rate according to PCWG3 criteria defined as the first occurrence of a 50% or more decline in PSA from baseline.

In any one of the methods provided herein, the method provides a partial (PR) or complete response (CR) based on RECIST 1.1 for soft tissue or PCWG3 for bone (PCWG3-modified RECIST 1.1).

In any one of the methods provided herein, the brief pain inventory pain intensity score decreased at 6 months post administration of I-131 1095 and enzalutamide. In one embodiment of any one of the methods provided herein, the brief pain inventory pain intensity score decreased at 6 months post administration of I-131 1095 and enzalutamide is any reduction on one or more pain scores.

In any one of the methods provided herein, the BSI index decreases from baseline post administration of I-131 1095 and enzaiutamide. In any one of the methods provided herein, the BSI index decreases from baseline post administration of I-131 1095 and enzalutamide is a decline of 5% or more, 10% or more, 20% or more or 50% or more.

In any one of the methods provided herein, overall and component scores of the Functional Assessment of Cancer Therapy-Prostate (FACT-P) questionnaire post administration of I-131 1095 and enzalutamide improved from baseline. In any one of the methods provided herein, overall and component scores of the Functional Assessment of Cancer Therapy-Prostate (FACT-P) questionnaire post administration of I-131 1095 and enzalutamide is improved from baseline by a 1 unit or more improvement of one or more components of FACT-P scores.

In any one of the methods provided herein, a EQ-5D-5L index post administration of I-131 1095 and enzalutamide increased from a baseline EQ-5D-5L index. In any one of the methods provided herein, a EQ-5D-5L index post administration of I-131 1095 and enzalutamide is increased from a baseline EQ-5D-5L index by at least 5 or more health score units based on a scale of zero (worst health) to 100 (best health).

In another aspect, a kit comprising one or more containers each containing one or more doses of I-131 1095 or components to produce I-131 1095, is provided. In any one of the kits provided herein, each dose of enzalutamide and/or each dose of I-131 1095 is/are any one of such doses provided herein. In any one of the kits provided herein, each dose of I-131 1095 is 75-100 mCi or the components are components to produce I-131 1095 in an amount of up to 200 mCi.

In any one of the kits provided herein, the kit comprises or further comprises one or more containers each comprising one or more doses of enzalutamide.

In any one of the kits provided herein, the kit comprises or further comprises one or more containers each comprising one or more doses of [18F]DCFPyL or components to produce [18F]DCFPyL.

In any one of the kits provided herein, the kit further comprises a diluent.

In any one of the kits provided herein, the kit further comprises instructions for radiolabeling any one or more or all of the compounds of the kit and/or instructions for administering any one or more or all of the compounds of the kit.

In any one of the kits provided herein, the kit further comprises patient prescribing information, such as for any one of the subjects provided herein, such as for the treatment of a metastatic castration-resistant prostate cancer patient who is prostate specific membrane antigen (PSMA)-avid, chemotherapy-naïve and progressed on abiraterone, such information including instructions for dosing and administration.

In any one of the kits provided herein, the kit further comprises instructions for producing any one or more or all of the compounds with the components.

In any one of the kits provided herein, the container containing one or more doses of I-131 1095 is provided within a lead shielded device.

### DETAILED DESCRIPTION OF THE INVENTION

The present invention relates, at least in part, to the surprising discovery of the effectiveness of I-131 1095 in combination with enzalutamide in the treatment of prostate cancer, particularly castration-resistant metastatic prostate cancer. It is thought that the combination therapy can overcome resistance developed to antiandrogen therapy, such as abiraterone, in conjunction with sensitizing cells to radiotherapy induced cell death.

Small molecule therapeutic, I-131 (iodine-131) 1095, binds to the extracellular domain of prostate specific membrane antigen (PSMA), a protein that is highly expressed in prostate cancer cells, and upon binding, internalized by the prostate cancer cells, where its I-131 beta radiation kills malignant cells. The ability to specifically deliver radiation to prostate cancer cells anywhere in the body allows a commonly used therapy (radiation) to be used with precision to attack systemic disease. Preclinical data has shown high tumor uptake and a favorable tumor to kidney discrimination yielding a lethal radiation dose to the tumor while minimizing normal tissue dose. In human prostate cancer mouse models, the compound, administered in single or multiple dose schedules, significantly reduced tumor burden for a prolonged period of time and enhanced survival with no significant signs of toxicity. When used in a compassionate use setting, I-131 1095 markedly reduced PSA levels and bone pain but was well tolerated in a group of heavily-pretreated advanced prostate cancer patients.

The chemical structure of I-131 1095 (or 131I 1095) (i.e., 311I-(S)-2-(3-((S)-1-carboxy-5-(3-(4-iodophenyl)ureido)pentyl)ureido)pentanedioic acid) is:

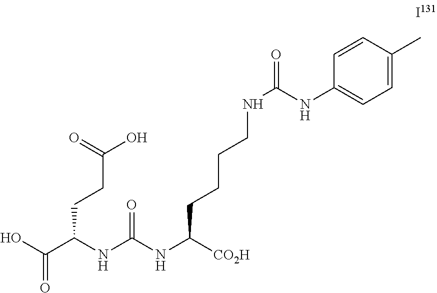 [SMILES: Cc1ccc(NC(=O)NCCCC[C@H](NC(=O)N[C@@H](CCC(=O)O)C(=O)O)C(=O)O)cc1]

Enantiomers, stereoisomers, rotamers, tautomers, diastereomers, or racemates thereof are also included in the definition of “I-131 1095”. U.S. Pat. No. 8,487,129 describes such compounds, which compounds and methods of their making are incorporated herein by reference. These compounds are for use in any one of the methods and compositions provided herein, in an embodiment.

An “antiandrogen,” as used herein, refers to an agent that blocks (e.g., inhibits) the action of androgen hormones and androgen-regulated molecules. Adrenergic receptor antagonists are herein considered to be antiandrogens. The term “antiandrogen” includes antiandrogens, antiandrogen analogs, and antiandrogen derivatives. In prostate cancer, antiandrogens block the activity of testosterone, which typically slows prostate cancer growth. In some embodiments, an antiandrogen blocks enzyme cytochrome P450 17A1, encoded by the CYPI7A gene. Antiandrogens may be steroidal or non-steroidal (also referred to as “pure”). Examples of antiandrogens include, without limitation, abiraterone (ZYTIGA®), enzalutamide (XTANDI®), nilutamide (NILANDRON®), flutamide (EULEXIN®), bicalutamide (CASODEX®), and orteronel (TAK-700, Tokai Pharmaceuticals, Inc.)

A subject provided herein is one with prostate cancer on which PSMA is or can be expressed. PSMA is a 100 kD Type II membrane glycoprotein expressed in prostate tissues (Horoszewicz et al., 1987, Anticancer Res. 7:927-935; U.S. Pat. No. 5,162,504). PSMA was characterized as a type II transmembrane protein having sequence homology with the transferrin receptor (Israeli et al., 1994, Cancer Res. 54:1807-1811) and with NAALADase activity (Carter et al., 1996, Proc. Natl. Acad. Sci. U.S.A. 93:749-753). PSMA is expressed in increased amounts in prostate cancer (Horoszewicz et al., 1987, Anticancer Res. 7:927-935; Rochon et al., 1994, Prostate 25:219-223; Murphy et al., 1995, Prostate 26:164-168; and Murphy et al., 1995, Anticancer Res. 15:1473-1479).

In one embodiment of any one of the methods provided herein, a subject has had prior antiandrogen therapy, such as with abiraterone. In another embodiment of any one of the methods provided herein, such subject has had prior antiandrogen therapy, such as with abiraterone, but not prior cytotoxic chemotherapy, such as with taxane chemotherapy. In another embodiment of any one of the methods provided herein, any one of such subjects has prostate cancer that has progressed despite these prior treatment(s). In another embodiment of any one of the methods provided herein, any one of such subjects is one with mCRPC that has progressed despite prior treatment(s).

In one embodiment of any one of the methods provided herein, a subject has had multiple rounds of prior antiandrogen therapy, such as with abiraterone. In another embodiment of any one of the methods provided herein, a subject has had prior antiandrogen therapy, such as with abiraterone, but not prior cytotoxic chemotherapy, such as with taxane chemotherapy. In another embodiment of any one of the methods provided herein, any one of such subjects has prostate cancer that has progressed despite the prior treatment(s). In another embodiment of any one of the methods provided herein, any one of such subjects is one with mCRPC that has progressed despite prior treatment(s).

“Progression”, as used herein, refers to prostate cancer cell proliferation that is not reduced, such as with a treatment, such as any one of the prior treatments or combinations thereof that are referred to herein, respectively. Disease progression may be indicated by rising PSA levels (e.g., an increase from baseline or a prior measurement of ≥25% and ≥2 ng/mL above nadir with or without a second such assessment of progression ≥3 weeks later), soft tissue disease progression as defined by RECIST 1.1, bone disease progression defined by two or more new lesions on bone scan, and/or new pain in an area of radiographically evident disease. In any one of the methods provided herein, progression is or has been determined with any one or more of the methods provided herein. In one embodiment of any one of the methods provided herein, prostate cancer that is progressing is not substantially inhibited by the prior treatment or combination thereof and would be considered non-responsive by a clinician.

In one embodiment of any one of the methods provided herein, the subject has or has had soft tissue or bone progression, such as with a scan that shows progression relative to a comparison scan performed during prior abiraterone therapy or after discontinuation from abiraterone. In one embodiment of any one of the methods provided herein, the subject has or has had soft tissue or bone progression, such as with a scan that shows progression relative to results from a previous scan, such as performed during prior abiraterone therapy or after discontinuation from abiraterone.

In any one of the methods provided herein, progression is or has been determined with any one of the methods provided herein.

In one aspect of any one of the methods provided herein, a method of treating any one of the subjects provided herein comprising administering one or more doses of I-131 1095 and one or more doses of enzalutamide is provided. In one embodiment of any one of the methods provided herein, any one of the methods can include a step of determining the tumor avidity in the subject prior to the administering of the one or more doses of I-131 1095 and the one or more doses of the enzalutamide. In one embodiment of any one of the methods provided herein, only subjects expressing PSMA-avid metastatic castration-resistant prostate cancer are treated with one or more doses of I-131 1095 and one or more doses of enzalutamide.

“Tumor PSMA avidity” is a measure of the tumor burden by imaging the level of PSMA on the prostate cancer cells. Notably, this measure can indicate the likelihood a subject will benefit from any one of the methods provided herein. Tumor avidity can be determined with [18F]DCFPyL, such as with PET/CT (i.e., [18F]DCFPyL PET/CT). PyL (also known as [18F]DCFPyL) is a fluorinated PSMA-targeted Positron Emission Topography (“PET”) imaging agent that enables visualization of metastases, such as bone and soft tissue metastases or both. Imaging with such an agent can be used to determine the presence or absence of recurrent and/or metastatic prostate cancer. U.S. Pat. No. 8,778,305 describes such a compound, which compound and methods of its making are incorporated herein by reference. The compound is for use in any one of the methods and compositions provided herein, in an embodiment.

In one embodiment of any one of the methods provided herein, the tumor avidity is determined with [18F]DCFPyL PET/CT. In another embodiment of any one of the methods provided herein, the tumor avidity is determined with [18F]DCFPyL PET/CT and if a significant increase in SUV count from baseline is determined, the subject is one for which the methods provided herein can have a benefit. Accordingly, in any one of the methods provided herein, the subject is such a subject, in another embodiment of any one of the methods provided herein, the subject for treatment is one in which the tumor avidity assessment using[18]DCFPyL, PET/CT indicates significant uptake (SUV>1.5×SUV of liver) in bone lesions and/or indicates significant uptake in visceral or lymph node lesions that have a long diameter of >2 cm. In one embodiment of any one of the methods provided herein, uptake is significant if this is observed in at least one lesion. Any one of the methods provided herein can include a step of assessing tumor avidity in the subject prior to and/or during treatment with the combination therapy as provided herein.

Efficacy of treatment of a subject treated according to any one of the methods provided herein can also be evaluated by assessing the prostate specific antigen (PSA) response rate according to Prostate Cancer Clinical Trials Working Group 3 (PCWG3) criteria defined as a confirmed 50% or greater decline from baseline, e.g., of I-131 1095 and enzalutamide compared to enzalutamide alone. Secondary endpoints can also be evaluated, including radiographic response based on Response Evaluation Criteria In Solid Tumors (RECIST), Progression Free Survival (PFS) and overall survival (OS). Tumor avidity may also be evaluated after any one of the treatments provided herein. Any one of the methods provided herein can include a step of evaluating any one or more of the endpoints provided herein.

Also provided herein are compositions (such as a kit), for example, pharmaceutical compositions, which comprise I-131 1095, enzalutamide or [18]DCFPyL. A composition, in some embodiments, includes a physiologically or pharmaceutically acceptable carrier, excipient, or stabilizer combined with any of the aforementioned compounds, or a combination thereof. As used herein, “pharmaceutically acceptable carrier” or “physiologically acceptable carrier” includes any and all salts, solvents, dispersion media, coatings, antibacterial and antifungal agents, isotonic and absorption delaying agents, and the like that are physiologically compatible. A “pharmaceutically-acceptable carrier,” as used herein, refers to one or more compatible solid or liquid tillers, diluents or encapsulating substances that are suitable for administration into a human. The term “carrier” denotes an organic or inorganic ingredient, natural or synthetic, with which the active ingredient is combined to facilitate the application. A carrier may be suitable for intravenous administration (e.g., by injection or infusion).

In some embodiments of any one of the methods provided herein, a composition may be administered to a subject in pharmaceutically-acceptable amounts and in pharmaceutically-acceptable compositions. The term “pharmaceutically acceptable” means a non-toxic material that does not interfere with the effectiveness of the biological activity of the active ingredients. Such compositions may contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded. “Administered” as used herein is direct or indirect administration (e.g., directing or prescribing to a subject a therapeutic where the subject themselves administers or takes the therapeutic as a result of the directing or prescribing).

In some embodiments of any one of the compositions or methods provided herein, a composition may conveniently be presented in unit dosage form and may be prepared by any of the methods well-known in the art of pharmacy. In some embodiments of any one of the compositions or methods provided herein, compositions are prepared by uniformly and intimately bringing the active compound into association with a liquid carrier, a finely divided solid carrier, or both, and then, if necessary, shaping the product.

Generally, the compositions are sterile. A composition can be administered by any conventional route, including injection or by gradual infusion over time. The administration may, for example, be intravenous. For enzalutamide compositions, the composition can be one for oral administration, such as in capsule form.

Compositions as provided herein, in some embodiments, may be in or administered in effective amounts in any one of the compositions or methods provided herein. An “effective amount” is that amount of an active compound that alone, or together with further doses or together with one or more other compounds, produces the desired response, e.g., inhibits cell proliferation of PSMA-expressing prostate cancer cells and/or kills PSMA-expressing prostate cancer cells. For cancer, this may involve only slowing the progression of a cancer, for example, temporarily, although more preferably, it involves halting the progression of the cancer permanently. This can be monitored by routine methods.

Also provided herein are kits comprising the composition(s). In some embodiments of any one of the kits provided, the kits comprise at least one container containing I-131 1095. In another embodiment of any one of the kits provided, the kit further contains enzalutamide. In another embodiment of any one of the kits provided, the kit further contains [18]DCFPyL or one or more compounds that can be used in a process for producing [18F]DCFPyL. In some embodiments of any one of the kits provided, the kit may comprise a carrier being compartmentalized to receive in close confinement therein one or more containers or series of containers such as test tubes, vials, flasks, bottles, syringes, or the like. The components of the kits can be packaged either in aqueous medium, etc. Any one of the kits provided herein may, in some embodiments, also comprise a diluent and/or instructions for radiolabeling and/or instructions for diluting aqueous components of the kits. The container(s) may be enclosed within a lead-shielded device for any one of the kits provided herein.

### EXAMPLES

### Example 1—A Multicenter, Randomized, Controlled Phase 2 Study: Efficacy and Safety of I-131-1095 Radiotherapy in Combination with Enzalutamide in Metastatic Castration-Resistant Prostate Cancer (mCRPC) Patients Who are ¹⁸F-DCFPyL Prostate-Specific Membrane Antigen (PSMA)-Avid, Chemotherapy-Naïve, and Progressed on Abiraterone (ARROW)

This study is a multicenter, open label, randomized phase 2 study of I-131-1095 radiotherapy (≤100 mCi/dose every 8 weeks for up to four doses) in combination with enzalutamide compared to enzalutamide alone in patients with progressive mCRPC. Patients have progression on abiraterone and indicated for treatment with enzalutamide. Patients have not had prior treatment with taxane-based chemotherapy.

Approximately 120 subjects receive I-131-1095 plus enzalutamide. Subjects undergo PSMA imaging with ¹⁸F-DCFPyL PET/CT to confirm high PSMA expression. All subjects are followed for one year following the first dose of treatment for the following assessments of prostate cancer: PSA, disease status on CT/MR, bone scan and ¹⁸F-DCFPyL-PET, automated bone scan index, SSE, survival status, and patient reported outcomes (PROs). The consensus guidelines of the Response Evaluation Criteria in Solid Tumors, version 1.1 (RECIST 1.1) and the Prostate Cancer Clinical Trials Working Group 3 (PCWG3) criteria are used to determine radiologic response and clinical and radiographic disease progression.

Safety and tolerability are assessed by the collection of treatment-emergent AEs, monitoring of vital signs and physical examinations, safety laboratory tests, and ECGs. Survival data, adverse events of special interest (AESIs) and new anti-cancer therapy are collected for one year following completion or early discontinuation of the treatment period.

Subjects who meet eligibility criteria receive 18F-DCFPyL and PET/CT to assess the randomization criterion. If subjects do not demonstrate PSMA tumor avidity based on central assessment of the protocol defined avidity criteria their total study duration is estimated to be up to 45 days. Subject who meet all eligibility and randomization criteria are randomized (2:1) to receive I-131-1095 plus enzalutamide or enzalutamide alone and have scheduled follow-up visits in the treatment period up to 12 months after their first dose of I-131-1095 and/or enzalutamide and for another 12 months thereafter for survival and safety follow-up. The total maximum study duration for randomized subjects is 25 months and 15 days.

The treatment period is comprised of 20 visits, including four I-131 1.095 dosing cycles (16 visits) and four additional safety/efficacy visits. A dosing cycle is defined as an 8-week period starting with Day 1 of dosing. The start of a dosing cycle corresponds with the day of study drug administration for subjects receiving I-131 1095. A delay in dosing beyond the 8-week cycle may occur up to an additional 6 weeks. I-131 1095 is administered intravenously at 100 mCi for the initial therapeutic dose, and up to 3 additional dose(s) between 75 mCi-100 mCi each, administered at least 8 weeks apart as determined by initial dosimetry evaluation and occurrence of dose-limiting events. Enzalutamide (Xtandi) is given orally once daily as prescribed by the physician as standard of care. Typically the dose is four 40 mg capsules (160 mg) daily.

### IP Doses and Mode of Administration:

There are two investigational products (IPs) in this study:

1) 18F-DCFPyL (PyL) for the imaging of prostate cancer lesions is administered to all subjects prior to randomization to confirm PSMA avidity in subjects randomized to treatment with I-131 1095. PyL is supplied to each institution on the planned day of administration in a unit-dose syringe (contained in a lead shield unit-dose system) with no additional preparation required for a 9 mCi (333 MBq) unit dose.

2) I-131 1095 for the PSMA-targeted treatment of prostate cancer is administered following randomization. Each shielded vial containing I-131-1095 is shipped frozen at −70° C. and stored at ≤−70° C. or thawed for immediate use. Each vial contains approximately 200 mCi of I-131-1095 at Time of Calibration (TOC). Aseptic procedures are used during withdrawal of study radiopharmaceutical for IV administration of a prescribed dose up to 100 mCi (e.g., 75-100 mCi).

### Diagnosis and Main Eligibility Criteria: Inclusion Criteria:

Subjects meet the following inclusion criteria:
1. Male ≥18 years of age
2. Histologically or cytologically confirmed adenocarcinoma of the prostate without neuroendocrine differentiation or small cell features at initial diagnosis
3. Castration-resistant prostate cancer, with serum testosterone ≤50 ng/dL (1.73 nM) at screening
4. Metastatic disease documented by bone lesions on whole body bone scan or soft tissue lesions measurable per RECIST 1.1 on CT/MRI prior to randomization or up to 21 days prior to screening
5. Evidence of disease progression on prior abiraterone therapy. Disease progression is defined by meeting at least one of the following criteria:
a. PSA progression as defined by a minimum of two rising PSA levels at least 1 week apart
b. Soft tissue disease progression* defined by RECIST 1.1
c. Bone disease progression* defined by two or more new lesions on bone scan *For subjects enrolling on the basis of soft tissue or bone progression, the baseline scan shows progression relative to a comparison scan performed during prior abiraterone therapy or after discontinuation from abiraterone. If the comparison scan is not available, the baseline scan report references the previous scan to document progression
   6. Planned for treatment with enzalutamide
   7. Subjects who are ineligible or choose not to receive taxane-based chemotherapy based on personal preference or physician opinion. Examples of conditions that could make a patient ineligible or refuse to receive taxane-based chemotherapy, but would allow them to still be eligible to receive I-131-1095 include the following:
a. Symptomatic peripheral neuropathy CTCAE Grade 2 or higher;
b. Clinically significant cardiovascular disease per the Investigator or treating physician
   8. Subjects receiving bisphosphonates must have been on stable doses for ≥4 weeks prior to randomization
   9. Eastern Cooperative Oncology Group (ECOG) performance status 0-2
   10. If sexually active, agree to use a medically acceptable method of birth control (e.g., spermicide in conjunction with a barrier such as a condom) or sexual abstinence from the time of dosing through 28 days after the last dose of I-131 1095. Sperm donation is prohibited from the time of dosing through 28 days after the last dose of I-131 1095. Female partners must use hormonal or barrier contraception unless postmenopausal or abstinent.
   11. Estimated life expectancy of at least 6 months as determined by the Investigator or treating physician
   12. Able and willing to provide signed informed consent and comply with protocol requirements

### Exclusion Criteria:

Subjects meeting any of the following exclusion criteria are not eligible for this study:
1. Received any anti-tumor therapy within 4 weeks of randomization, with the exception of abiraterone, gonadotropic-releasing hormone (GnRH) therapy and non-radioactive bone-targeted agents
2. Received prior chemotherapy for prostate cancer
3. Superscan as evidenced on baseline bone scan
4. Treatment with Strontium-89, Samarium-153, Rhenium-186, Rhenium-188, Radium-223 within 6 months prior to randomization
5. Prior hemi-body irradiation. Prior external beam radiotherapy to >25% of bone marrow
6. Prior PSMA-targeted radioligand therapy
7. Major surgery within 4 weeks of randomization
8. Impaired organ function as evidenced by the following laboratory values in Screening labs:
a. Absolute neutrophil count <1500 μL
b. Platelet count <100,000/μL
c. Hemoglobin <9.5 g/dL
d. Albumin <3.0 g/dL (30 g/L)
e. Total bilirubin >2×ULN unless in instances of known or suspected Gilbert's disease
f. AST and ALT>2.5×ULN
g. Serum creatinine >1.5×ULN or Calculated creatinine clearance (CrCL)<30 mL/min (Cockroft-Gault equation), or currently on renal dialysis
   9. Hypothyroidism as evidenced by Screening TSH>3.0 mIU/L with confirmed low Free T3 (<230 ng/dL) or Free T4 (<0.7 ng/dL)
   10. QT interval corrected for heart rate (QTc)>470 msec during screening
   11. Previous use of enzalutamide for >7 days prior to consent
   12. Planned initiation of alternative therapy for prostate cancer, investigational therapy, or participation in clinical trials during the study
   13. History or risk of seizure (i.e., clinically significant neurological disorder) or any other condition that contraindicates treatment with enzalutamide (Xtandi®) as per the package insert
   14. Active malignancy other than prostate cancer, with the exception of curatively treated non-melanoma skin cancer, carcinoma in situ, or non-muscle invasive bladder/urothelial cancer
   15. Subjects with any medical condition or other circumstances that, in the opinion of the investigator, compromise obtaining reliable data, achieving study objectives, or completing the study.

### Randomization Criterion:

Subjects are also screened for 18F-DCFPyL avidity as defined by the below criteria to proceed to Randomization:
18F-DCFPyL PET/CT imaging shows significant PSMA uptake (SUN/max>1× liver SUVmean) in at least one lesion (all lesions in one embodiment), except as noted below:
PSMA negative soft tissue lesions <1.0 cm in short axis;
PSMA negative lymph node lesions <1.5 cm in short axis;
PSMA negative bone lesions with a soft tissue component <1.0 cm in short axis or without a soft tissue component of any size

All subjects are followed for one year following the first dose of randomized treatment for the following assessments of prostate cancer: PSA, disease status on CT/MR, bone scan and 18F-DCFPyL-PET, automated bone scan index, SSE, survival status, and patient reported outcomes (PROs). The consensus guidelines of the Response Evaluation Criteria in Solid Tumors, version 1.1 (RECIST 1.1) and the Prostate Cancer Clinical Trials Working Group 3 (PCWG3) criteria are used by investigators to determine radiologic response and clinical and radiographic disease progression.

### Efficacy Study Endpoints:

Primary Endpoint: PSA response rate according to PCWG3 criteria defined as the first occurrence of a 50% or more decline in PSA from baseline, confirmed by a second measurement at least 3 weeks later.

Secondary Endpoints:
Objective response rate (ORR) from baseline to the final assessment performed for each patient defined as the proportion of patients who have a partial (PR) or complete response (CR) based on RECIST 1.1 for soft tissue or PCWG3 for bone (PCWG3-modified RECIST 1.1)
PFS defined as time from randomization to the first occurrence of radiographic progression based on RECIST 1.1 for soft tissue or PCWG3-modified RECIST 1.1 for bone, respectively, or protocol defined unequivocal clinical progression, or death on study from any cause
OS defined as time from randomization to death from any cause
PSA progression defined as the time from randomization to the date of the first PSA increase from baseline >25% and >2 ng/mL above nadir confirmed by a second PSA assessment defining progression >3 weeks later per PCWG3
rPFS defined as the dine from randomization to first occurrence of radiographic progression based on PCWG3-modified RECIST 1.1
Duration of response defined as the time from the first date of complete or partial response to the first occurrence of radiographic progression based on PCWG3-modified RECIST 1.1, or protocol defined unequivocal clinical progression.
Time to next treatment defined as the time from randomization to initiation of any new treatment of prostate cancer

Exploratory Efficacy Endpoints:
18F-DCFPyL uptake defined as change from baseline in SUV Change in baseline in 18F-DCFPyL positive lesion counts
Time from randomization to first symptomatic skeletal event (SSE) defined as symptomatic fracture, radiation or surgery to the bone, or spinal cord compression
Rate of pain progression, defined as an increase of >30% from baseline in the Brief Pain Inventory Short Form (BPI-SF) pain intensity score at 6 months
Change from baseline in the physical domain from FACT-P
Change from baseline in the social/family domain from FACT-P
Change from baseline in the emotional domain from FACT-P
Change from baseline in the functional/well-being domain from FACT-P
Change from baseline in overall and component scores of the Functional Assessment of Cancer Therapy-Prostate (FACT-P) questionnaire
Changes from baseline in SF-12v2 domain scores, Physical Component Summary (PCS) scores and Mental Component Summary (MCS) scores
Summary statistics for EQ-5D-5L VAS
Summary statistics for EQ-5D-5L Index
Changes from baseline in aBSI
Change from baseline in ECOG performance status

### Assessment of Efficacy

### PSA (Total)

PSA (total) will be assessed throughout the study at intervals.

### Radiographic Assessments

Subjects will undergo CT/MRI, whole-body bone scans and ¹⁸F-DCFPyL PET/CT at intervals or at any time progression is suspected. The assessment of radiographic response and progression will be performed using RECIST 1.1 for measurable soft tissue disease on CT/MRI and PCWG3 for bone disease on bone scan. Only patients with either presentation at baseline will be included in the respective assessment. The same imaging modality should be used throughout the study for any given patient. Radiographic imaging is not required after radiographic progression has been confirmed and documented.

Tumor burden based on ¹⁸F-DCFPyL PET/CT will be assessed at a central core imaging lab based on SUV, lesion counts, tumor volume and total lesion PSMA expression. Total lesion PSMA expression will be calculated by multiplying tumor volume and mean SUV,

### Bone Scan

A radionuclide bone scan (either ⁹⁹mTc or ¹⁸F-NaF PET/CT, depending upon the site's standard of care) will be obtained at intervals. If conducted as part of Screening, ¹⁸F-NaF PET bone scans must be done at least five physical half-lives (10 hours) prior to ¹⁸F-DCFPyL injection. Bone scans will be assessed by the Investigator for radiographic response and progression for PSMA avidity and aBSI assessments.

### CT or MRI

A contrast-enhanced (if not contraindicated) CT or MRI of the abdomen and pelvis and a CT of the chest will be obtained at intervals. High density oral contrast medium (oral water contrast is acceptable) cannot be administered within 5 days prior to study drug injection.

¹⁸F-DCFPyL (PyL) PET/CT imaging will be performed at intervals. All PyL PET/CT scans should be submitted in DIACOM format to evaluate tumor avidity, changes from baseline in PyL uptake as defined by total SUV counts and PyL-positive lesion counts.

Automated Bone Scan Index (aBSI)

Subject's whole body bone scans will be assessed at intervals to determine changes over time. The Bone Scan Index (BSI) is defined as the percentage of total skeletal mass occupied by bone metastases. aBSI (automated BSI) is software for automatically and semi-automatically estimating BSI from whole-body planar bone scans.

### Radiographic Progression

Radiographic progression of bone disease per PCWG3 is defined as the appearance of 2 or more new bone lesions on first post-treatment scan, with at least 2 additional new lesions seen on the next, confirmatory scan. If at least 2 additional new lesions are seen on the confirmatory scan, the date of progression is the date of the first post-treatment scan, when the first two new bone lesions were identified. For all other scans after the first post-treatment scan, progression is defined as the appearance of at least two new lesions when compared to the first post-treatment scan, and then confirmed on a subsequent scan. The date of progression is the date of the scan that the first documents at least two new lesions.

Radiographic progression of nodal and visceral disease per PCWG3-modified RECIST 1.1 is defined as at least a 20% increase in the sum of diameters of target lesions, using the smallest sum on the study as reference. The sum of diameters of target lesions must also be an absolute increase of at least 5 mm. The appearance of one or more new lesions is also considered progression.

### Unequivocal Clinical Progression

Unequivoval clinical progression is defined when the subject is no longer deemed to be benefitting (NCLB) from treatment with I-131 1095 and any of the following occur:
a) New onset of prostate cancer pain requiring chronic opiate use (chronic opiate use (excluding acetaminophen-opiate fixed-dose combinations) will be considered as daily use for more than 7 consecutive days or more than 10 days within a 14-day period), or
b) Deterioration of ECOG performance status to ≥3 as a result of prostate cancer, or
c) Initiation of cytotoxic chemotherapy for prostate cancer, or
d) Radiation therapy or surgical intervention because of complications of tumor progression. Palliative radiotherapy of symptoms due to prostate cancer will not be considered unequivocal progression and will not mandate study drug discontinuation.

### Symptomatic Skeletal Event (SSE)

SSEs will be collected as Adverse Events and are defined as symptomatic fracture, radiation, or surgery to the bone, or spinal cord compression. Asymptomatic fractures are skeletal-related events and not considered SSEs of clear clinical significance.

### Brief Pain Inventory—Short Form (BPI-SF)

The Brief Pain Inventory questionnaire is a validated instrument that is a patient self-rated scale assessing level of pain, effect of the pain on activities of daily living, and analgesic use. The short form of the Brief Pain Inventory (BPI-SF) used in this study can be found in APPENDIX A. Another example can be found at npcrc.org/files/news/briefpain_short.pdf. Any such questionnaires can be used in any one of the methods provided herein.

### Functional Assessment of Cancer Therapy-Prostate (FACT-P)

The FACT-P quality of life (QoL) questionnaire is a multi-dimensional, self-reported QoL instrument specifically designed for use with prostate cancer patients. It consists of 27 core items which assess patient function in four domains: physical, social/family, emotional and functional well-being, which is further supplemented by 12 site-specific items to assess for prostate-related symptoms. Each item is rated on a 0 to 4 Likert-type scale, and then combined to produce subscale scores for each domain, as well as a global quality of life score with higher scores representing better QoL. See APPENDIX B, APPENDIX E, and facit.org/FACITOrg/Questionnaires for example FACT-P questionnaires. Any such questionnaires can be used in any one of the methods provided herein.

### EQ-5Q-5L

The EQ-5D-5L consist of a 5-item questionnaire and the EQ Visual Analogue scale (EQ VAS). The descriptive system comprises 5 dimensions (mobility, self-care, usual activities, pain/discomfort, anxiety/depression.) Each dimension has 5 levels: no problems, slight problems, moderate problems, severe problems, and extreme problems. The respondent is asked to indicate his health state by ticking (or placing a cross) in the box against the most appropriate statement in each of the 5 dimensions. This decision results in a 1-digit number expressing the level selected for that dimension. The digits for 5 dimensions can be combined in a 5-digit number describing the respondent's health state. It should be noted that the numerals 1-5 have no arithmetic properties and should not be used as a cardinal score. See APPENDIX C for EQ-5D-51. Other examples of EQ-5D guides can be found at euroqol.org/publications/user-guides. Any such questionnaires can be used in any one of the methods provided herein.

### ST-12v2 Health Survey

The SF-12v2 Health Survey is a 12-item general health survey which can be self-administered or interview-administered. The survey measures the eight health domains (physical functioning, role-physical, bodily pain, general health, vitality, social functioning, role-emotional, mental health). Together these provide psychometrically-based physical component summary (PCS) and mental component summary (MCS) scores. See APPENDIX D for the SF-12v2 Health Survey. See also Maruish, M. E. (Ed.). (2012). User's manual for the SF-12v2 Health Survey (3rd ed.). Lincoln, R.I.: QualityMetric Incorporated for other examples. Any such surveys can be used in any one of the methods provided herein.

1. A method of treating a subject with prostate specific membrane antigen (PSMA)-avid prostate cancer, the method comprising:
administering to a subject one or more doses of I-131 1095 and administering one or more doses of enzalutamide, wherein the subject's tumor PSMA avidity, prior to the administering of the one or more doses of I-131 1095 and the administering of the one or more doses of enzalutamide, was assessed.

2. The method of claim 1, wherein the method further comprises assessing tumor avidity in the subject prior to the administering of the one or more doses of I-131 1095 and the administering of the one or more doses of enzalutamide.

3. The method of claim 1, wherein the tumor avidity was/is assessed with [18F]DCFPyL PET/CT.

4. The method of claim 3, wherein the [18F]DCFPyL PET/CT indicates significant uptake (SUVmax>1×SUVmean of liver) in at least one lesion except as follows:
(a) PSMA negative soft tissue lesions <1.0 cm in short axis,
(b) PSMA negative lymph node lesions <1.5 cm in short axis; and
(c) PSMA negative bone lesions with a soft tissue component <1.0 cm in short axis or without a soft tissue component of any size.

5. The method of claim 3, wherein the [18F]DCFPyL PET/CT indicates significant uptake (SUV>1.5×SUV of liver) in bone lesions and/or indicates significant uptake in visceral or lymph node lesions that have a long diameter of >2 cm.

6-7. (canceled)

8. The method of claim 1, wherein the subject meets one or more or all of the following:
(a) has castration-resistant prostate cancer with serum testosterone ≤50 ng/dL (1.73 nM),
(b) has metastatic disease documented by bone lesions on whole body bone scan or soft tissue lesions measurable per RECIST 1.1 on CT/MRI,
(c) had disease progression on prior abiraterone therapy, optionally, wherein disease progression on prior abiraterone therapy is defined by meeting at least one of the following:
(i) PSA progression defined by a minimum of two rising PSA levels at least 1 week apart,
(ii) soft tissue disease progression defined by RECIST 1.1, and
(iii) bone disease progression defined by two or more new lesions on bone scan,
(d) did not receive prior taxane-based chemotherapy,
(e) had prior treatment with bisphosphonates and on stable doses for ≥4 weeks prior, and/or
(f) Eastern Cooperative Oncology Group (ECOG) performance status 0-2.

9. (canceled)

10. The method of claim 1, wherein the subject has not/does not:
(a) received any anti-tumor therapy within 4 weeks prior,
(b) received prior chemotherapy for prostate cancer,
(c) received treatment with Strontium-89, Samarium-153, Rhenium-186, Rhenium-188, Radium-223 within 6 months prior,
(d) had a prior hemi-body irradiation or prior external beam radiotherapy to >25% of bone marrow,
(e) had a prior PSMA-targeted radioligand therapy,
(f) have impaired organ function, optionally, wherein the impaired organ function is:
(i) absolute neutrophil count <1500 μL,
(ii) platelet count <100,000/μL,
(iii) hemoglobin <9.5 g/dL,
(iv) albumin <3.0 g/dL (30 g/L),
(v) total bilirubin >2×ULN,
(vi) AST and ALT>2.5×ULN, and
(vii) serum creatinine >1.5×ULN or calculated creatinine clearance (CrCL)<30 mL/min (Cockroft-Gault equation) or on renal dialysis, and/or
(g) have hypothyroidism, optionally, wherein hypothyroidism is TSH≥3.0 mIU/L with low Free T3 (<230 ng/dL) or Free T4 (<0.7 ng/dL).

11-12. (canceled)

13. The method of claim 1, wherein the subject has PSMA-avid metastatic castration resistant prostate cancer (mCRPC) and/or disease progression on prior antiandrogen therapy.

14. (canceled)

15. The method of claim 13, wherein the prior antiandrogen therapy is prior abiraterone therapy.

16. (canceled)

17. A method of treating a chemotherapeutic-naïve subject whose castration-resistant prostate cancer has progressed despite abiraterone treatment, the method comprising:
determining PSMA-avidity of the cancer using a radiolabeled PSMA-binding agent;
administering to a subject having a PSMA-avid cancer one or more doses of I-131 1095 and
administering one or more doses of enzalutamide.

18. (canceled)

19. The method of claim 17, wherein the PSMA-avidity is determined in the subject prior to the administering of the one or more doses of I-131 1095 and the administering of the one or more doses of enzalutamide.

20-22. (canceled)

23. The method of claim 1, wherein each dose of I-131 1095 is between 75-100 mCi and/or wherein each dose of enzalutamide is four 40 mg capsules.

24. The method of claim 23, wherein each dose of I-131 1095 is administered intravenously.

25. The method of claim 23, wherein the I-131 1095 dose is administered optionally, every 8 weeks, or every 12 weeks and/or wherein each dose of enzalutamide is administered orally once a day.

26-28. (canceled)

29. The method of claim 1, wherein the method further comprises assessing the subject prior to the administering of the one or more doses of I-131 1095 and the administering of the one or more doses of enzalutamide, the assessing comprising:
(a) determining the subject has castration-resistant prostate cancer with serum testosterone ≤50 ng/dL (1.73 nM),
(b) determining the subject has metastatic disease documented by bone lesions on whole body bone scan or soft tissue lesions measurable per RECIST 1.1 on CT/MRI,
(c) determining the subject has disease progression on prior abiraterone therapy, optionally, wherein disease progression on prior abiraterone therapy is defined by meeting at least one of the following:
(i) PSA progression defined by a minimum of two rising PSA levels at least 1 week apart,
(ii) soft tissue disease progression defined by RECIST 1.1, and
(iii) bone disease progression defined by two or more new lesions on bone scan,
(d) determining the subject has not had prior taxane-based chemotherapy,
(e) determining the subject has had prior treatment with bisphosphonates and on stable doses for ≥4 weeks prior, and/or
(f) determining the subject has Eastern Cooperative Oncology Group (ECOG) performance status 0-2.

30. (canceled)

31. The method of claim 1, wherein the method further comprises assessing the subject prior to the administering of the one or more doses of I-131 1095 and the administering of the one or more doses of enzalutamide, the assessing comprising:
(a) determining the subject has not received any anti-tumor therapy within 4 weeks prior,
(b) determining the subject has not received prior chemotherapy for prostate cancer,
(c) determining the subject has not received treatment with Strontium-89, Samarium-153, Rhenium-186, Rhenium-188, Radium-223 within 6 months prior,
(d) determining the subject has not had a prior hemi-body irradiation or prior external beam radiotherapy to >25% of bone marrow,
(e) determining the subject has not had a prior PSMA-targeted radioligand therapy,
(f) determining the subject does not have impaired organ function, and/or
(g) determining the subject has not have hypothyroidism.

32-33. (canceled)

34. A kit comprising:
one or more containers each containing one or more doses of I-131 1095 or components to produce I-131 1095.

35-41. (canceled)

42. Method of treatment management for a subject with PSMA-avid metastatic, castration resistant prostate cancer and cancer progression on prior abiraterone therapy comprising:
a) demonstrating the subject's tumor PSMA avidity in bone lesions (SUV>1.5 SUV of liver) and/or visceral or lymph node lesions that have a long diameter of >2 cm, and
b) providing a treatment of one or more doses of I-131 1095 and one or more doses of enzalutamide.

43-47. (canceled)

48. The method of claim 1, wherein the method provides:
(i) a PSA response rate according to PCWG3 criteria defined as the first occurrence of a 50% or more decline in PSA from baseline, and/or
(ii) a partial (PR) or complete response (CR) based on RECIST 1.1 for soft tissue or PCWG3 for bone (PCWG3-modified RECIST 1.1).

49. (canceled)

50. The method of claim 1, wherein:
(i) the brief pain inventory pain intensity score decreased at 6 months post administration of I-131 1095 and enzalutamide,
(ii) the BSI index decreases from baseline post administration of I-131 1095 and enzalutamide,
(iii) overall and component scores of the Functional Assessment of Cancer Therapy-Prostate (FACT-P) questionnaire post administration of I-131 1095 and enzalutamide improved from baseline, and/or
(iv) a EQ-5D-5L index post administration of I-131 1095 and enzalutamide increased from a baseline EQ-5D-5L index.

51-53. (canceled)
